(19) United States
(12) Patent Application Publication
(10) Pub. No.: US 2004/0193263 A1
(43) Pub. Date: Sep. 30, 2004

(54) IOL and assembly

(76) Inventor: Philip L. Bryan, Honeoye Falls, NY (US)

Correspondence Address: Bausch & Lomb Incorporated, One Bausch & Lomb Place, Rochester, NY, 14604-2701, US

(21) Appl. No.: 10/402,231

(22) Filed: Mar. 27, 2003

(51) Int. Cl. A61F 2/16; A61F 9/007

(52) U.S. Cl. 623/6.12; 623/6.18; 606/107

(57) ABSTRACT

An intraocular lens and an assembly for implanting an IOL into an eye wherein the IOL is provided with first and second truncated edges which engage longitudinal channels formed along opposite sides of an inserter lumen. The truncated edges cause the IOL to maintain a preferred rotational orientation within the inserter lumen thereby minimizing the chance of IOL damage due to unintentional rotation of the IOL as it is being passed through the lumen.

### BACKGROUND OF THE INVENTION

The present invention relates to intraocular lenses (IOLs), and more particularly relates to IOLs and IOL inserter assemblies designed to control the rotational orientation of the IOL as it is passed through an inserter and into an eye.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye. IOLs are sometimes also implanted within an eye where the natural lens remains intact (phakic eye).

In each of the above-described surgical procedures, the surgeon cuts an incision into the cornea wherethrough the IOL is passed and implanted within the eye. Various instruments and methods for implanting the IOL in the eye are known. In one method, the surgeon simply uses surgical forceps having opposing blades which are used to grasp the IOL and insert it through the incision into the eye. While this method is still practiced today, more and more surgeons are using more sophisticated IOL inserter devices which offer advantages such as affording the surgeon more control when inserting the IOL into the eye. IOL inserter devices have recently been developed with reduced diameter insertion tips which allow for a much smaller incision to be made in the cornea than is possible using forceps alone. Smaller incision sizes (e.g., less than about 3mm) are preferred over larger incisions (e.g., about 3.2 to 5+mm) since smaller incisions have been attributed to reduced postsurgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL inserter device must therefore be designed in such a way as to permit the easy passage of the IOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Thus, as explained above, the IOL inserter device must be designed to permit easy passage of the IOL therethrough. It is equally important that the IOL be expelled from the tip of the IOL inserter device and into the eye in a predictable orientation and manner. Should the IOL be expelled from the tip in the wrong orientation, the surgeon must manipulate the IOL in the eye which could result in trauma to the surrounding tissues of the eye. It is therefore highly desirable to have a inserter device which will pass and expel the IOL from the inserter device tip and into the eye in a controlled, predictable and repeatable manner.

To ensure controlled expression of the IOL through the tip of the IOL inserter device, the IOL must first be loaded into the IOL inserter device. The loading of the IOL into the inserter device is therefore also a precise and very important step in the process. Incorrect loading of an IOL into the inserter device is oftentimes cited as the reason for a failed IOL delivery sequence.

In a typical IOL inserter device, the IOL inserter utilizes a plunger having a tip which engages the IOL (which has been previously loaded and compressed into the inserter lumen) to pass it through the inserter lumen. The IOL thus interfaces with the plunger tip as well as the lumen of the inserter device. These component interfaces are dynamic in the sense that the forces between the interfacing components may vary as the IOL is pushed through the lumen. Control of these dynamic forces is therefore of utmost importance or otherwise the IOL may be damaged during delivery. For example, should the IOL be free to twist and/or turn as it is moved through the inserter, the force between the IOL and the plunger tip and/or the inserter lumen may uncontrollably increase to the point of IOL damage.

Various inserter devices have been proposed which attempt to address these problems, yet there remains a need for an IOL inserter and method which delivers the IOL into an eye in a controlled and predictable manner and which at the same time will not damage the IOL.

### SUMMARY OF THE INVENTION

The present invention provides an IOL and an assembly including an IOL and inserter device which are complimentarily designed in a manner which determines and controls the dynamic interface between the IOL and inserter components as the IOL is pushed through the inserter device and into an eye. As such, the chances of a failed IOL delivery due to damage caused by the IOL delivery sequence is minimized or eliminated.

The invention is primarily directed at an IOL inserter device in which the IOL is compressed laterally within the lumen thereof. Such a device may be seen in U.S. Pat. No. 5,944,725 which is of common ownership with the instant invention. In this type of inserter device, the opposite edges 16a,16b of the compressed IOL body 14 are engaged within opposite longitudinal channels 92,94 of the inserter lumen 107 as seen in FIGS. 6A-6C thereof. As the plunger tip 36 engages and pushes the IOL body through the inserter lumen, the edges ride along channels 92,94. However, since the IOL body is essentially round and symmetrical, the IOL may unexpectedly begin to rotate about its optical axis (which extends perpendicular to the longitudinal channels), causing increased delivery forces and the chance of IOL damage caused thereby. This may happen, for example, if the plunger tip engages the IOL body laterally of the longitudinal axis of the lumen.

The IOL is designed in a manner substantially preventing the uncontrolled rotation of the IOL about its optical axis as it is pushed through the inserter lumen. The IOL body is designed with a particular edge geometry which will engage the lumen channels only when the IOL is in a specific rotational orientation. The complimentary shape of the IOL edges and the longitudinal channels control the rotational orientation of the IOL as it is passed through the inserter lumen, regardless of whether the plunger engages the IOL laterally off-set of the lumen longitudinal axis. Furthermore, should the IOL be initially positioned in a rotationally off-set manner, upon initial pushing of the IOL with the plunger, the dynamic interface between the IOL edge geometry and the lumen channels will cause the IOL to seek the preferred rotational position. This rotational position will thus be automatically found and maintained as the IOL is pushed through and out of the inserter device.

### BRIEF DESCRIPTION OF THE DRAWING

FIG. 1 is a plan view of a prior art IOL;

FIG. 2 is a plan view of an IOL according to a first embodiment of the present invention;

FIG. 3 is a plan view of an IOL according to a second embodiment of the present invention;

FIG. 4 is a partial, longitudinal, cross-sectional view showing a prior art IOL being pushed through an inserter device by a plunger;

FIG. 5 is a partial, longitudinal, cross-sectional view showing an IOL according to the present invention loaded into an inserter device and showing the IOL in the preferred rotational position within the inserter lumen;

FIG. 6 is a cross-sectional view as taken along the line 6-6 of FIG. 5 showing the IOL in the compressed condition ready for delivery through the inserter device.

### DETAILED DESCRIPTION

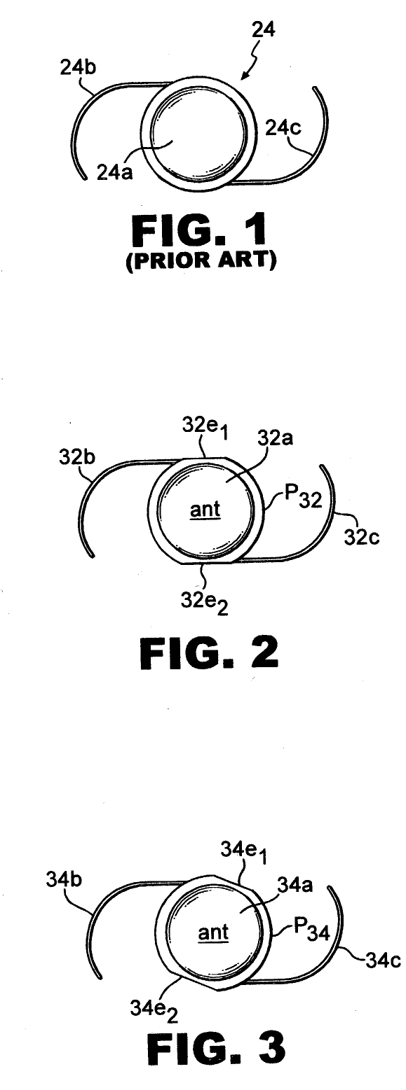

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens or IOL such as prior art IOL 24 seen in FIG. 1. IOLs may also be placed in an eye where the natural lens remains intact (termed a “phakic eye”). This may be done to improve a person's vision where other vision correction means are not wanted or appropriate for the patient, for example. The IOL may be placed in the eye in a position which is forward, or more typically, inside the eye's lens capsule which is located behind the iris in the posterior chamber of the eye.

An IOL includes a central optic portion 24a which simulates the extracted natural lens by directing and focusing light upon the retina, and further includes means for securing the optic in proper position within the capsular bag. A common IOL structure for securing the optic is called a haptic which is a resilient structure extending radially outwardly from the periphery of the optic. In a particularly common IOL design, two haptics 24b, 24c extend from opposite sides of the optic and curve to provide a biasing force against the inside of the capsule which secures the optic in the proper position within the eye.

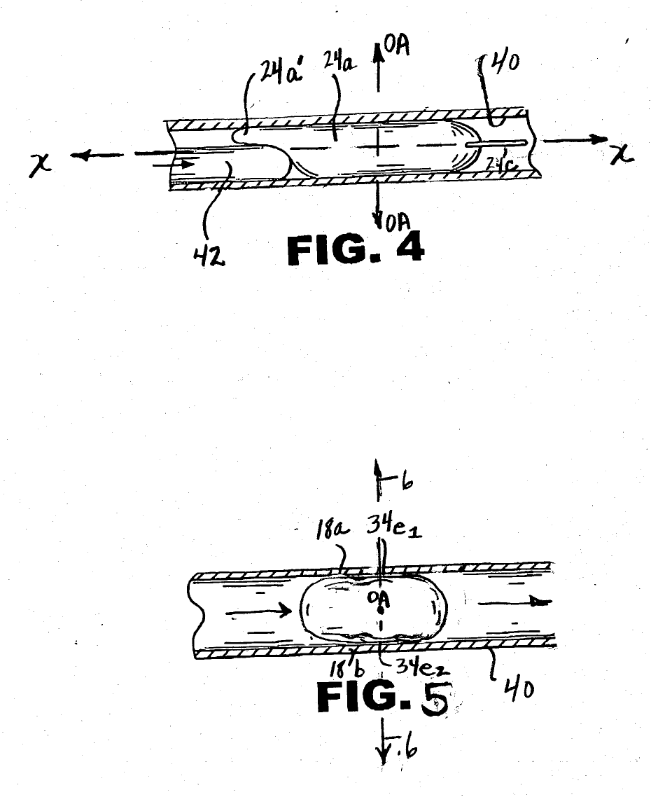

Referring now to FIGS. 2 and 3, two embodiments of the inventive IOL 32, 34 are shown, respectively. Both IOL 32 and 34 include a central optic portion 32a, 34a having opposite anterior (ant32a, ant34a) and posterior surfaces (the posterior surfaces cannot be seen), respectively, defined by a peripheral wall P₃₂, P₃₄. When implanted within the eye, anterior optic surface ant32a and ant34a faces the cornea and the respective opposite posterior optic surface faces the retina. A pair of haptics 32b,c and 34b,c are attached to and extend from opposite sides of the peripheral wall P₃₂, P₃₄ of optic portion 32a, 34a, respectively. The haptics are configured to provide a biasing force against the interior of the eye to properly position IOL 32, 34 therein. In typical IOL designs, the engagement between the haptics and interior eye creates a biasing force causing the implanted IOL optic 32a, 34a to vault posteriorly toward the retina. In the case where the IOL is implanted in the lens capsule, the posterior surface of the IOL optic presses tightly against the interior of the posterior capsule wall to prevent posterior capsular opacification, or PCO. It is noted that any other known IOL positioning means (e.g., closed loop haptics or plate haptics, etc.) are possible and within the scope of the invention. Furthermore, IOL 32, 34 may be made from any suitable IOL material, e.g., PMMA, silicone, hydrogels and composites thereof, etc.

There are a several ways in which IOL may be implanted into an eye. One currently popular method is to use an inserter device having a lumen into which the IOL is loaded and compressed to allow the IOL to be inserted through a relatively small incision in the eye (e.g., 3 mm or less). Once the IOL is expressed from the inserter into the eye, it assumes its original shape due to the elastic nature of the material from which the IOL is formed (see discussion above). The inserter device also includes a plunger having a plunger tip which engages the IOL to advance the IOL through the lumen. The surgeon manually operates and controls advancement of the plunger and thus also the IOL through the lumen.

FIG. 4 shows a prior art IOL 24 compressed within an inserter lumen 40 and engaged by a plunger tip 42. As explained in the Background section hereof, it is very important that the IOL delivery sequence go as smoothly as possible to prevent damage to the delicate IOL. FIG. 4 illustrates a potential problem with a delivery sequence. In this case, the plunger tip 42 has engaged the IOL optic 24a in a location which is laterally off-set from the central longitudinal axis x-x of the inserter lumen 40. In the situation, the IOL optic 24a begins to rotate about its optical axis OA resulting in portions of the IOL optic 24a becoming engaged between the plunger tip 42 and lumen wall 40 as indicated at 24a′. This results in an increase in the drag forces between the IOL, lumen wall and plunger tip which may very likely cause damage to the IOL and should thus be avoided.

To solve this problem of unintentional lens rotation within the inserter lumen, the present invention provides an IOL having truncated edges which will interface with the lumen wall to cause the IOL to maintain this preferred rotational position. It is of course understood that the truncated edges are positioned and formed so as to not interfere with the optical functioning of the IOL, nor adversely affect placement and ongoing presence of the IOL within the eye.

In a first embodiment shown in FIG. 2, IOL optic 32 includes first and second truncated edges 32e₁ and 32e₂ which extend substantially parallel to each other along opposite sides of the optic peripheral wall P₃₂. In this embodiment, the truncated edges 32e₁ and 32e₂ are positioned adjacent the attached ends of haptics 32b,32c, respectively, and extend generally parallel thereto. In the embodiment of FIG. 3, the first and second truncated edges 34e₁ and 34e₂ extend substantially parallel to each other along opposite sides of peripheral P₃₄, but are further spaced from the attached ends of haptics 34b,34c than in the embodiment of FIG. 2.

The exact placement of the truncated edges with respect to the haptics may vary, however, bench testing has indicated the embodiment of FIG. 3 may perform better than the embodiment of FIG. 2 when used with the inserter design of U.S. Pat. No. 5,944,725. In the embodiment of FIG. 3, truncated edges 34ₑ₁ and 34ₑ₂ extend at an angle relative to the attached end of the respective haptic 34b,34c.

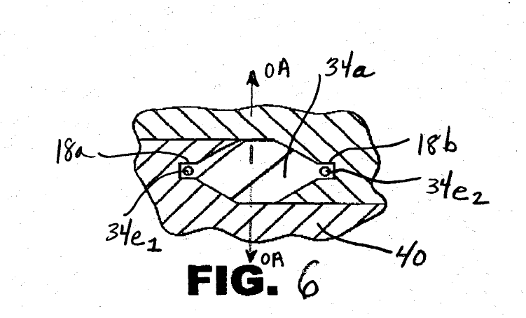

More particularly, FIG. 6 herein illustrates the cross-section of IOL 34a in the laterally compressed state within inserter lumen 40. Lumen 40 includes opposite longitudinal channels 18a,18b in which opposite edges 34e₁ and 34e₂ of the optic peripheral wall P engage. As the IOL is pushed by the plunger through the lumen, the IOL optic edges 34ₑ₁ and 34ₑ₂ ride along within channels 18a,18b. Absent the present invention of truncated edges, the IOL optic is free to rotate about its optical axis OA which is undesirable as explained above with regard to FIG. 4. By providing truncated edges, IOL 34 will maintain a preferred rotational position as the IOL travels through the lumen. Thus, the chance IOL damage caused by unintentional rotation of the IOL is therefore minimized or eliminated.

Thus, as seen in FIG. 5., as the IOL 34 is pushed by the plunger (not shown) in the direction of the linear arrows, edges 34ₑ₁ and 34ₑ₂ engage and remain within channels 18a, 18b, respectively. The dynamics of the parallel interface between the truncated edges and the lumen channels are such that the IOL will resist any rotational movement about the lens optical axis OA, even if a destabilizing force is applied to the lens, e.g., a laterally off-set force being applied thereto by the plunger. As such, the chance of IOL damage caused by unintentional lens rotation within the lumen is minimized or eliminated.

Although the invention has been described with regard to preferred embodiments thereof, it is understood that variations may be made thereto. For example, instead of being substantially straight, the truncated edges and channel walls may assume any other suitable, cooperative configurations such as curved. The first and second truncated edges may be of the same shape or dissimilar shapes. Additionally, the first and second truncated edges may be located at any distance or angle with respect to the haptics, and may further be of the same or different angular orientations.

What is claimed is:

1. An intraocular lens having an optic defined by a peripheral wall and first and second truncated edges formed opposite each other in said peripheral wall.

2. The intraocular lens of claim 1 and further comprising first and second haptics secured to and extending from said peripheral wall adjacent said first and second truncated edges, respectively.

3. The intraocular lens of claim 1 wherein said truncated edges are substantially straight.

4. An assembly for implanting an intraocular lens into an eye, said assembly comprising:
a) an intraocular lens having an optic defined by a substantially circular peripheral wall and first and second truncated edges formed opposite each other in said peripheral wall; and
b) an inserter device in which said intraocular lens may be loaded and compressed for passage therethrough and into said eye, said inserter device having a lumen including first and second longitudinally extending channels in which said first and second truncated edges engage when said IOL is loaded therein.

5. The intraocular lens of claim 4 and further comprising first and second haptics secured to and extending from said peripheral wall adjacent said first and second truncated edges, respectively.

6. The intraocular lens of claim 4 wherein said truncated edges are substantially straight.

7. The intraocular lens of claim 4 wherein said truncated edges are curved.
